(19) United States
(12) Patent Application Publication
(10) Pub. No.: US 2015/0247185 A1
(43) Pub. Date: Sep. 3, 2015

(54) DEVICE AND METHOD FOR PURIFYING TARGET SUBSTANCE

(71) Applicant: Seiko Epson Corporation, Tokyo (JP)

(72) Inventors: Toshiro MURAYAMA, Fujimi (JP); Fumio TAKAGI, Chino (JP)

(21) Appl. No.: 14/632,286

(22) Filed: Feb. 26, 2015

(30) Foreign Application Priority Data
Feb. 28, 2014 (JP) 2014-038183

(51) Int. Cl. C12Q 1/68

(52) U.S. Cl. CPC C12Q 1/6806

(57) ABSTRACT

A target substance purification device includes: a mount for installing a capillary containing a liquid plug and having a longitudinal direction; a magnetic force applying mechanism that applies a magnetic force to the capillary to retain a magnetic body detachably retaining the target substance; a control mechanism that restricts movement of the magnetic body along the longitudinal direction of the capillary; and a liquid sending mechanism that moves the liquid along the longitudinal direction of the capillary while the movement of the magnetic body is being restricted by the control mechanism.

### BACKGROUND

1. Technical Field

The present invention relates to a device and a method for purifying a target substance.

2. Related Art

Boom et al. reports a method for conveniently extracting nucleic acids from biological materials with the use of a nucleic acid-binding solid phase support such as silica particles, and a chaotropic agent (J. Clin. Microbiol., vol. 28 No. 3, p. 495-503 (1990)). The method of Boom et al., and all the other extraction methods that involve adsorbing a nucleic acid on a support with a nucleic acid-binding solid phase support (such as silica) and a chaotropic agent basically include the steps of (1) adsorbing a nucleic acid on a nucleic acid-binding solid phase support in the presence of a chaotropic agent (adsorbing step), (2) washing the support and the adsorbed nucleic acid with a washing liquid to remove the non-specifically bound foreign substances and the chaotropic agent (washing step), and (3) eluting the nucleic acid from the support with water or a low salt concentration buffer (eluting step), as described, for example, in JP-A-11-146783, and JP-A-2009-207459.

A problem of such methods, however, is that each support particle is a very fine magnetic particle, and its hydrophilic surface may cause some of the particles to remain in the aqueous solution eluent when the externally applied magnetic procedures are insufficient. The presence of such magnetic particle residues in the eluent may interfere with the subsequent detection of a reaction by blocking the excitation light applied for detecting an amplification reaction.

### SUMMARY

An advantage according to some aspects of the invention is to provide a device and a method for purifying a target substance whereby magnetic particles can be efficiently removed.

An aspect of the invention is directed to a device for purifying a target substance, including: a mount for installing a capillary containing a liquid plug and having a longitudinal direction; a magnetic force applying mechanism that applies a magnetic force to the capillary to retain a magnetic body detachably retaining the target substance; a control mechanism that restricts movement of the magnetic body along the longitudinal direction of the capillary; and a liquid sending mechanism that moves the liquid along the longitudinal direction of the capillary while the movement of the magnetic body is being restricted by the control mechanism.

Another aspect of the invention is directed to a device for purifying a target substance, including: a capillary containing a liquid plug and having a longitudinal direction; a magnetic force applying mechanism that applies a magnetic force to the capillary to retain a magnetic body detachably retaining the target substance; a control mechanism that restricts movement of the magnetic body along the longitudinal direction of the capillary; and a liquid sending mechanism that moves the liquid along the longitudinal direction of the capillary while the movement of the magnetic body is being restricted by the control mechanism.

In both of the aspects of the invention described above, the target substance may be a nucleic acid, and the capillary may include: a first plug of oil; a second plug of a washing liquid that undergoes phase separation from the oil, and washes the magnetic body detachably retaining the nucleic acid; a third plug of oil; a fourth plug of an eluent that undergoes phase separation from the oil, and elutes the nucleic acid from the magnetic body detachably retaining the nucleic acid; and a fifth plug of oil, the first plug, the second plug, the third plug, the fourth plug, and the fifth plug being contained inside the capillary in this order. The device may include a moving mechanism that moves the magnetic body within a plane that crosses the longitudinal direction of the capillary. The magnetic force applying mechanism may include a permanent magnet. The liquid sending mechanism may include a pressure applying member, and the pressure applying member may be a plunger. Alternatively, the liquid sending mechanism may include a suction member, and the suction member may be a vacuum pump. Alternatively, the capillary may have ends with detachable sealing members.

Still another aspect of the invention is directed to a method for purifying a target substance, including: introducing a magnetic body detachably retaining the target substance into a capillary having a liquid plug; externally applying a magnetic force to the capillary to retain the magnetic body inside the liquid; eluting the target substance from the magnetic body inside the liquid; and moving the liquid with the eluted target substance out of the capillary by moving the liquid along the longitudinal direction of the capillary while restricting movement of the magnetic body.

In the method according to this aspect of the invention, the target substance may be a nucleic acid, and the capillary may include: a first plug of oil; a second plug of a washing liquid that undergoes phase separation from the oil, and washes the magnetic body detachably retaining the nucleic acid; a third plug of oil; a fourth plug of an eluent that undergoes phase separation from the oil, and elutes the nucleic acid from the magnetic body detachably retaining the nucleic acid; and a fifth plug of oil, the first plug, the second plug, the third plug, the fourth plug, and the fifth plug being contained inside the capillary in this order, wherein the magnetic body detachably retaining the target substance may be introduced into the capillary from an end closer to the first plug, wherein the magnetic body may move into the eluent under a magnetic force externally applied to the capillary, wherein the target substance may elute from the magnetic body in the eluent, and wherein the eluent with the eluted target substance may move to an end closer to the fifth plug, and may discharge from the capillary at the end closer to the fifth plug. The method may include moving the magnetic body within a plane that crosses the longitudinal direction of the capillary. The liquid with the eluted target substance may be moved along the longitudinal direction of the capillary by applying pressure inside the capillary, and the pressure may be applied with a plunger. Alternatively, the liquid with the eluted target substance may be moved along the longitudinal direction of the capillary by creating suction inside the capillary, and the suction may be created with a vacuum pump. Alternatively, the capillary may have detachable sealing members at the both ends, and the capillary may be vertically held, and the liquid with the eluted target substance may be moved along the longitudinal direction of the capillary by opening and closing the sealing members.

The invention has thus enabled providing a device and a method for purifying a target substance whereby magnetic particles can be efficiency removed.

### BRIEF DESCRIPTION OF THE DRAWINGS

The invention will be described with reference to the accompanying drawings, wherein like numbers reference like elements.

FIGS. 1A and 1B are schematic diagrams of a nucleic acid purification device according to an embodiment of the invention.

FIG. 2 is a magnified view near a plug inside a capillary according to the embodiment of the invention.

FIG. 3 is a schematic diagram of the nucleic acid purification device with a reaction vessel and a substance extraction unit according to the embodiment of the invention.

FIGS. 4A and 4B are schematic diagrams representing a use of the nucleic acid purification device according to the embodiment of the invention.

FIGS. 5A to 5F are schematic diagrams representing a specific procedure of using the nucleic acid purification device according to the embodiment of the invention.

### DESCRIPTION OF EXEMPLARY EMBODIMENTS

The objects, features, advantages, and ideas of the invention will be clearly understood by a skilled person from the descriptions of the invention, and it would be easy for a skilled person to reproduce the invention from the following descriptions. The embodiments and concrete examples of implementation discussed in the following detailed explanation serve solely to illustrate or describe the preferred embodiments of the invention, and do not limit the invention in any ways. It will be understood that various modifications and alterations may be made to the invention by a skilled person from the following descriptions, provided such modifications and alterations do not exceed the intent and the scope of the invention disclosed below.

### Target Substance Purification Device

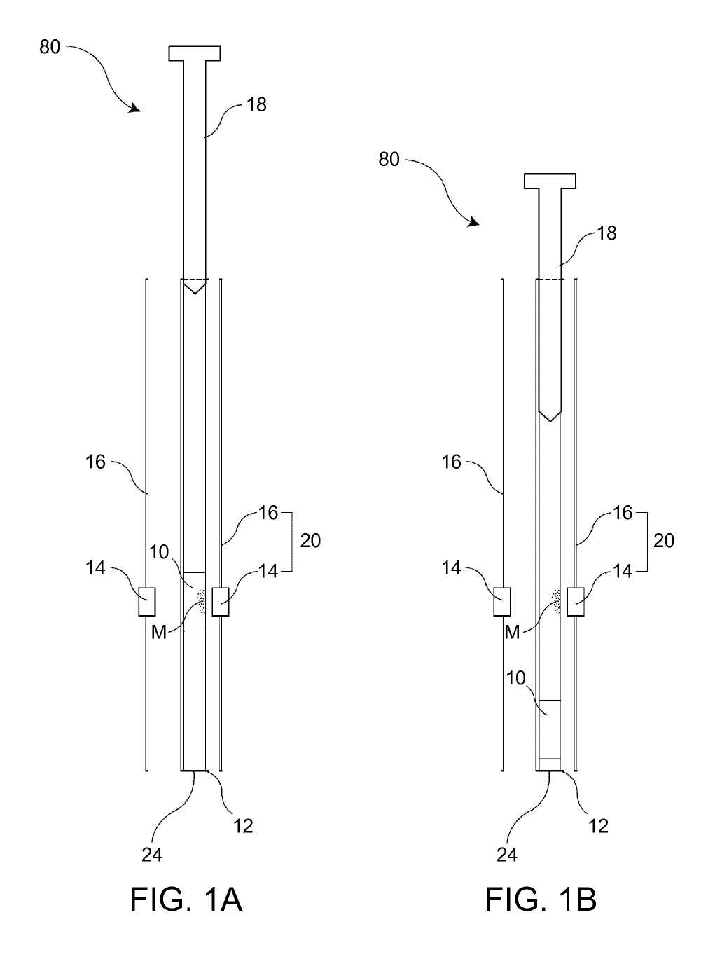

As illustrated in FIG. 1A, an embodiment of a target substance purification device 80 according to the invention includes a capillary 12 having a liquid plug 10, a magnetic body M detachably retaining a target substance inside the capillary 12, a magnetic force applying mechanism 14 that applies a magnetic force to the capillary 12 to retain the magnetic body, a control mechanism 16 that stops the longitudinal movement of the magnetic body inside the capillary 12, and a liquid sending mechanism 18 that moves a liquid along the longitudinal direction of the capillary 12 while the control mechanism 16 is stopping the movement of the magnetic body M.

The liquid plug 10 is preferably an aqueous solution, more preferably a salt solution, particularly a buffer. The capillary 12 may have more than one plug. In this case, it is preferable to provide a wax plug or an oil plug between the liquid plugs so that the liquid plugs can independently exist.

The capillary 12 is hollow inside, and has a tubing portion (also called “tube portion”) that allows a liquid to longitudinally travel inside the hollow space. The tube portion has a longitudinal direction, but may be bent. The size and the shape of the hollow space inside the tube portion are not particularly limited, as long as the liquid can maintain the form of a plug inside the tube portion. The size of the hollow space inside the tube portion, and the shape of the cross section perpendicular to the longitudinal direction may vary along the longitudinal direction of the tube portion. Whether the liquid can maintain a plug shape inside the tube portion depends on conditions such as the material of the tube portion, and the type of the liquid, and as such the shape of the cross section perpendicular to the longitudinal direction of the tube portion is appropriately designed to allow the liquid to maintain a plug shape inside the tube portion. The outer cross sectional shape of the tube portion perpendicular to the longitudinal direction is not limited either. The thickness of the tube portion is not particularly limited. When the cross section of the hollow space inside the tube portion perpendicular to the longitudinal direction is circular in shape, the tube portion may have an inner diameter (the diameter of the circle of the cross section of the inner hollow perpendicular to the longitudinal direction) of, for example, 0.5 mm to 3 mm. Such an inner diameter of the tube portion is preferable in terms of ease of forming a liquid plug over a wide range of tube portion materials and liquid types. The material of the tube portion is not particularly limited, and may be, for example, glass, polymer, or metal. The materials selected for the tube portion are preferably materials that are transparent to visible light, such as glass and polymer, because such materials provide visual access to inside of the tube portion (the hollow space) from outside. It is also preferable to use a magnetically transparent material or a non-magnetic material for the tube portion because such materials make it easier to externally apply a magnetic force to the tube portion such as when passing magnetic particles M through the tube portion.

The target substance is not limited, and may be a macromolecule such as a nucleic acid (e.g., DNA, RNA), and a protein, or a low-molecular substance such as a compound. The shape of the magnetic body M is not particularly limited, and is preferably particulate. In order to detachably retain the target substance, the magnetic body M either detachably binds to the target substance, or has a target substance-binding substance that detachably binds to the magnetic body M. For example, when the target substance is a nucleic acid, the magnetic body M may have nucleic acid binding molecules such as silica particles. The magnetic body M may have an antibody binding molecule such as protein A when the target substance is an antibody.

The purification device 80 according to the invention includes a magnetic force applying section 20 provided with the magnetic force applying mechanism 14 and the control mechanism 16. The magnetic force applying mechanism 14 externally applies a magnetic force to the capillary 12 to retain the magnetic body M inside the capillary 12, and control the movement of the magnetic body M. The magnetic force applying mechanism 14 can be designed by a skilled person with ease. For example, the magnetic force applying mechanism 14 has a magnetic force applying body such as a permanent magnet, and can retain the magnetic body M with the magnetic force of the magnetic force applying body. The control mechanism 16 controls the longitudinal movement of the magnetic body M inside the capillary 12, and can stop the magnetic body M as desired. This is made possible, for example, with a mechanism by which the magnetic force applying mechanism 14 can be moved, and stopped at the desired position. Preferably, the control mechanism 16 is adapted to move the magnetic body M within a plane that crosses the longitudinal direction of the capillary 12, particularly a plane orthogonal to the longitudinal direction of the capillary 12. Specifically, it is preferable that the control mechanism 16 swings the magnetic body M. For example, because the magnetic body M is attracted by whichever of the magnetic force applying bodies is closer to the magnetic body M, the magnetic force applying body that is more proximate than the other to the capillary attracts the magnetic body M when two magnetic force applying bodies are provided. As the magnetic force applying body is moved away from the capillary 12, the magnetic body M is attracted by the other magnetic force applying body approaching the capillary 12 from the opposite side. In this way, the magnetic body M can be moved sideways. The magnetic body M can thus be moved back and forth sideways by swinging the pair of magnetic force applying bodies sideways. This makes it easier for the magnetic body M to contact the liquid as it moves in the liquid plug 10, and improves the washing and eluting effects.

The liquid sending mechanism 18 of the purification device 80 is provided to move the liquid plug 10 along the longitudinal direction of the capillary 12, and eject the liquid through a capillary end portion 24. For example, the liquid sending mechanism 18 may be a pressure applying member that applies pressure to the liquid inside the capillary 12, and moves the liquid under the applied pressure, or a suction member that moves the liquid by evacuating the capillary 12 and creating a negative pressure therein. The pressure applying member may be, for example, a piston or a plunger as shown in FIGS. 1A and 1B. The suction member may be, for example, a vacuum pump. Preferably, the diameter of the piston or the plunger is the same as the inner diameter of the capillary 12, and the piston or the plunger is directly fitted to the capillary 12. In this case, as illustrated in FIG. 1B, the liquid plug can be moved along the longitudinal direction of the capillary 12 by pushing the piston or the plunger into the capillary 12. In the case of a vacuum pump, for example, the capillary 12 may be fitted to the opening of a side arm flask through a rubber stopper, and a vacuum may be created inside the flask with a vacuum pump such as an aspirator connected to the arm. The liquid plug also can be moved along the longitudinal direction of the capillary 12 in this manner. Alternatively, a detachable sealing member such as a cap may be provided at the both ends of the capillary 12, and may be opened and closed to move the liquid with the eluted target substance along the longitudinal direction of the capillary 12 under the force of gravity, and discharge and collect the liquid from the capillary 12 held vertical to the ground. It is also possible to use the capillary 12 provided at one end with a high volatility liquid plug that does not instantaneously mix with oil. Heating the plug after sealing this end of the capillary 12 vaporizes the high volatility liquid, and moves the liquid with the eluted target substance toward the opposite end from the high volatility liquid plug along the longitudinal direction of the capillary 12.

### Target Substance Purification Method

The purification device 80 of the foregoing configuration may be used to purify a target substance. For example, a target substance of interest for purification is obtained from a sample such as cells and viruses containing the target substance, for example, by lysing or extracting the sample with a lysing solution or an extractant. The magnetic body M that can detachably retain the target substance is then added, and bound to the target substance. The magnetic body M detachably retaining the target substance may be washed with a buffer or the like with a centrifuge tube or the like. The magnetic body M detachably retaining the target substance is then introduced into the capillary 12 having the liquid plug 10. The method for introducing the magnetic body M is not particularly limited, and the magnetic body M may be introduced into the capillary 12 through the end portion 24 after being suspended in the same liquid used for the liquid plug 10 so that the liquids coalesce inside the capillary 12.

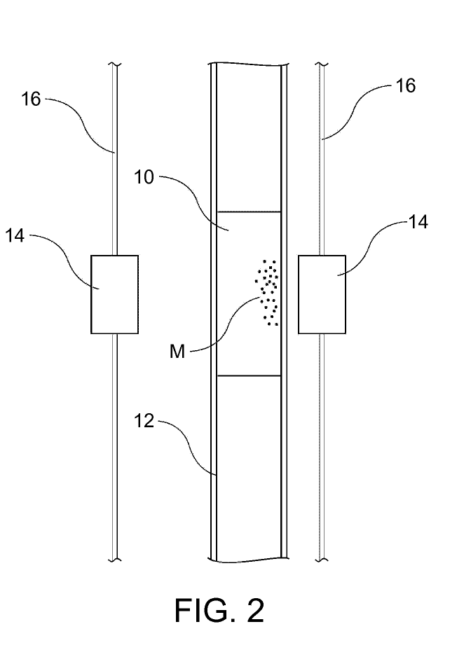

Simultaneously, a magnetic force is applied from outside of the capillary 12 to retain the magnetic body M inside the liquid. For example, as illustrated in FIG. 2, the magnetic body M may be retained inside the liquid by installing the magnetic force applying mechanism 14 in proximity outside (right side) of the liquid plug 10. The magnetic body M may be moved as desired inside the capillary 12, or may be retained at the specific position along the longitudinal direction of the capillary 12 by moving the magnetic force applying mechanism 14 along the longitudinal direction of the capillary 12, or by stopping the magnetic force applying mechanism 14 as desired with the control mechanism 16 that controls the movement of the magnetic force applying mechanism 14.

With the magnetic body M retained inside the liquid, the target substance is eluted from the magnetic body Min the liquid. The elution method is not particularly limited, and may be appropriately decided according to the manner in which the target substance binds to the target substance-binding substance of the magnetic body M. For example, in the case of a nucleic acid and silica, the liquid may be heated by heating the capillary. In the case of an antibody and an antigen, the antibody and the antigen may be separated from each other by making the pH acidic by addition of an acid to the liquid. Here, the liquid effect on the magnetic body M can be improved by swinging the magnetic body M on a plane that crosses the longitudinal direction of the capillary 12, particularly a plane orthogonal to the longitudinal direction of the capillary 12, as described above.

Thereafter, as illustrated in FIG. 1B, the liquid with the eluted target substance is moved along the longitudinal direction of the capillary 12, and ejected through the end 24 of the capillary 12 while stopping the longitudinal movement of the magnetic body M along the capillary 12. The liquid can then be collected to obtain the target substance that eluted from the magnetic body.

More than one liquid plug may be provided, one being a washing liquid for washing the magnetic body M detachably retaining the target substance, and the other being an eluent for eluting the target substance from the magnetic body M. Specifically, in this case, the capillary 12 may include a first plug of oil, a second plug of a washing liquid that undergoes phase separation from the oil, and washes the magnetic body detachably retaining the target substance, a third plug of oil, a fourth plug of an eluent that undergoes phase separation from the oil, and elutes the target substance from the magnetic body M detachably retaining the target substance, and a fifth plug of oil, in this order inside the capillary 12. As with the case of a single plug, the magnetic body M detachably retaining the target substance is introduced into the washing liquid. The magnetic body M is then moved to the eluent with the control mechanism 16 that controls the movement of the magnetic force applying mechanism 14. The target substance is then eluted in the eluent, and is ejected and collected through the end of the capillary as described above. Here, the washing effect can be further improved by providing more than one washing liquid plug.

### Example of Nucleic Acid Purification

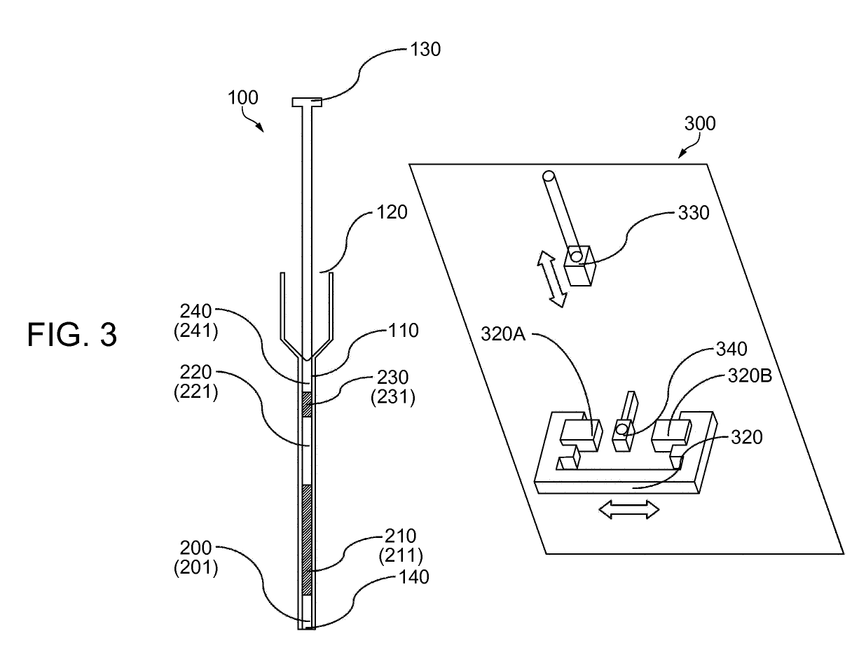

As illustrated in FIG. 3, the nucleic acid purification device of this Example includes a reaction vessel 100, and a substance extraction unit 300. The reaction vessel 100 includes a tube 110 having a longitudinal direction, and a plunger 130 connected to the tube 110 on the side of an opening 120 provided at one end of the tube 110. The tube 110 has an opening 140 at the end opposite the opening 120, and is charged with a first plug 200 of an oil, a second plug 210 of an washing liquid, a third plug 220 of an oil, a fourth plug 230 of an eluent, and a fifth plug 240 of an oil, in this order from the opening 140 toward the opening 120. The washing liquid and the eluent are aqueous solutions immiscible to the oil. The substance extraction unit 300 includes a mount 320 for installing the reaction vessel 100, permanent magnets 320A and 320B for applying a magnetic force to the side surface of the tube 110 of the reaction vessel 100, a liquid sending mechanism 330 that pushes the plunger 130 to send the liquid inside the reaction vessel 100, and a heating section 340 for heating a part of the tube 110. In this Example, silicone oil was used as the oil. A 76 mass % guanidine hydrochloride aqueous solution was used as the washing liquid. Sterile water was used as the eluent.

The following describes how the nucleic acid purification device is used to extract a nucleic acid from human blood.

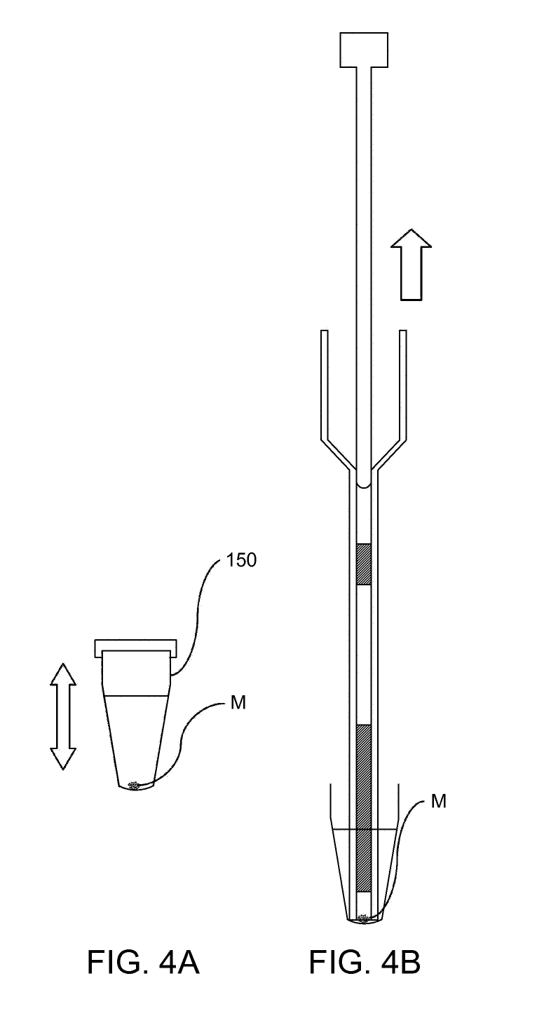

First, as illustrated in FIGS. 4A and 4B, 375 μL of an adsorbent, and 1 μL of a magnetic particle (magnetic particle M) dispersed liquid were contained in a 3-mL polyethylene vessel (adsorption vessel 150). The composition of the adsorbent was 76 mass % guanidine hydrochloride, 1.7 mass % EDTA.2Na dihydrate, and a 10 mass % polyoxyethylene sorbitan monolaurate aqueous solution (MagExtractor-Genome-, NPK-1; Toyobo). The magnetic particle dispersed liquid contained 50 volume % magnetic silica particles, and 20 mass % lithium chloride.

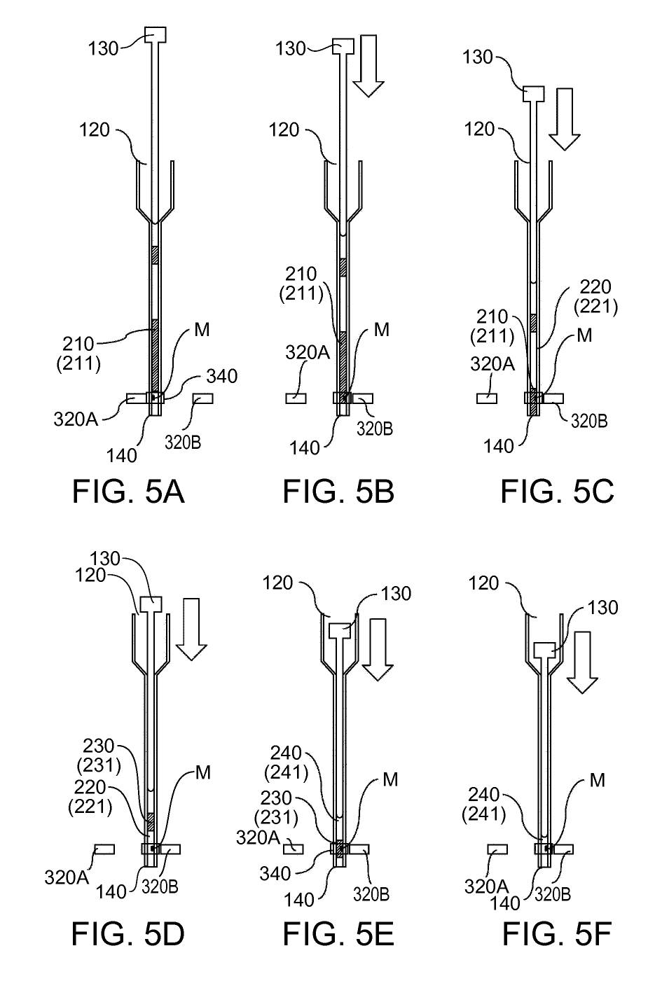

Fifty microliters of blood collected from human was pipetted into the adsorption vessel 150 through the opening. After capping the opening, the adsorption vessel 150 was agitated for 30 seconds by shaking it with hand to adsorb the blood nucleic acid to the magnetic particles M (see FIG. 4A). After removing the cap from the adsorption vessel 150, the opening 140 on the first plug side of the reaction vessel 100 is inserted into the adsorption vessel 150, and the plunger 130 was slid up against the opening 120. This charges the magnetic particles M inside the adsorption vessel 150 into the tube portion 110 of the reaction vessel 100 with the adsorbent (see FIG. 4B). The tube 110 with the plunger 130 is then installed on the mount 320 provided in the substance extraction unit 300. Here, the tube 110 is installed so that the opening 120 side of the tube is vertically above the opening 140. With the reaction vessel 100 installed in place, the opening 140 is past below the permanent magnets 320A and 320B, and the permanent magnets 320A and 320B are on the sides of the tube 110 at the first plug position. As a result, the magnetic force from the permanent magnets 320A and 320B moves the magnetic particles M to the first plug position inside the tube 110 (see FIG. 5A). The permanent magnet 320A and the permanent magnet 320B are on the opposite sides of the tube 110, facing each other with a certain distance in between. The permanent magnets 320A and 320B move reciprocally along the axis orthogonal to the longitudinal direction of the tube so as to vary their distances from the tube 110.

The liquids inside the tube are moved by pushing the plunger 130 toward the opening 140 while moving the permanent magnets 320A and 320B reciprocally along the axis orthogonal to the tube longitudinal direction. Under the applied pressure of the plunger 130 inside the tube, the second plug 210 of a washing liquid moves to the level that lies on the axis along which the magnetic force applying section is undergoing reciprocal movement. This moves the magnetic particles M to the second plug relative to the first plug of oil. The magnetic particles M are washed by the washing liquid, and the foreign substances around the magnetic particles M are removed as the permanent magnets 320A and 320B make reciprocal movement while the plunger 130 sends the liquid (see FIGS. 5B and 5C). As the plunger 130 sends the liquids further, the second plug moves toward the opening 140, and the third plug 220 of the oil comes to lie on the axis along which the permanent magnets 320A and 320B are undergoing reciprocal movement. It is desirable to stop the reciprocal movement of the permanent magnets 320A and 320B while the magnetic particles are passing through the interface between the second plug 210 and the third plug 220 so that the magnetic particles can make passage through the interface while the permanent magnet 320A or 320B is held still proximate to the tube side surface. In this way, it is possible to avoid the magnetic particles M from entering the third plug while the washing liquid forming the second plug is still present in abundance around the magnetic particles M (see FIGS. 5B and 5D). As the plunger 130 sends the liquids further, the third plug moves to the opening 140, and the fourth plug 230 forming the eluent comes to lie on the axis along which the permanent magnets 320A and 320B are undergoing reciprocal movement. The sending of the liquids is suspended upon the magnetic particles M having moved to the position near the middle of the fourth plug relative to the tube longitudinal direction, and the permanent magnets 320A and 320B are moved reciprocally while heating the eluent with the heating section 340 from the tube side surface to elute the adsorbed nucleic acid from the magnetic particles M into the eluent 230 (see FIG. 5E). This completes the elution of the nucleic acid into the eluent for amplification reaction. The plunger 130 is then pushed toward the opening 140 to discharge the eluent 230 with the eluted nucleic acid through the opening 140 (see FIG. 5F), and this liquid is charged into an amplification reaction vessel with a part of or all of the oil forming the third plug 220 and the fifth plug 240. In this way, the introduction of the liquid with the eluted extract into the amplification reaction vessel can be automated.

The entire disclosure of Japanese Patent Application No. 2014-038183, filed Feb. 28, 2014 is expressly incorporated by reference herein.

What is claimed is:

1. A device for purifying a target substance, comprising:
a mount for installing a capillary containing a liquid plug and having a longitudinal direction;
a magnetic force applying mechanism that applies a magnetic force to the capillary to retain a magnetic body detachably retaining the target substance;
a control mechanism that restricts movement of the magnetic body along the longitudinal direction of the capillary; and
a liquid sending mechanism that moves the liquid along the longitudinal direction of the capillary while the movement of the magnetic body is being restricted by the control mechanism.

2. A device for purifying a target substance, comprising:
a capillary containing a liquid plug and having a longitudinal direction;
a magnetic force applying mechanism that applies a magnetic force to the capillary to retain a magnetic body detachably retaining the target substance;
a control mechanism that restricts movement of the magnetic body along the longitudinal direction of the capillary; and
a liquid sending mechanism that moves the liquid along the longitudinal direction of the capillary while the movement of the magnetic body is being restricted by the control mechanism.

3. The device according to claim 1,
wherein the target substance is a nucleic acid, and
wherein the capillary includes:
a first plug of oil;
a second plug of a washing liquid that undergoes phase separation from the oil, and washes the magnetic body detachably retaining the nucleic acid;
a third plug of oil;
a fourth plug of an eluent that undergoes phase separation from the oil, and elutes the nucleic acid from the magnetic body detachably retaining the nucleic acid; and
a fifth plug of oil,
the first plug, the second plug, the third plug, the fourth plug, and the fifth plug being contained inside the capillary in this order.

4. The device according to claim 2,
wherein the target substance is a nucleic acid, and
wherein the capillary includes:
a first plug of oil;
a second plug of a washing liquid that undergoes phase separation from the oil, and washes the magnetic body detachably retaining the nucleic acid;
a third plug of oil;
a fourth plug of an eluent that undergoes phase separation from the oil, and elutes the nucleic acid from the magnetic body detachably retaining the nucleic acid; and
a fifth plug of oil,
the first plug, the second plug, the third plug, the fourth plug, and the fifth plug being contained inside the capillary in this order.

5. The device according to claim 1, comprising a moving mechanism that moves the magnetic body within a plane that crosses the longitudinal direction of the capillary.

6. The device according to claim 2, comprising a moving mechanism that moves the magnetic body within a plane that crosses the longitudinal direction of the capillary.

7. The device according to claim 1, wherein the magnetic force applying mechanism includes a permanent magnet.

8. The device according to claim 1, wherein the liquid sending mechanism includes a pressure applying member.

9. The device according to claim 8, wherein the pressure applying member is a plunger.

10. The device according to claim 1, wherein the liquid sending mechanism includes a suction member.

11. The device according to claim 10, wherein the suction member is a vacuum pump.

12. The device according to claim 1, wherein the capillary has both ends with detachable sealing members.

13. A method for purifying a target substance, comprising:
introducing a magnetic body detachably retaining the target substance into a capillary having a liquid plug;
externally applying a magnetic force to the capillary to retain the magnetic body inside the liquid;
eluting the target substance from the magnetic body inside the liquid; and
moving the liquid with the eluted target substance out of the capillary by moving the liquid along the longitudinal direction of the capillary while restricting movement of the magnetic body.

14. The method according to claim 13,
wherein the target substance is a nucleic acid, and
wherein the capillary includes:
a first plug of oil;
a second plug of a washing liquid that undergoes phase separation from the oil, and washes the magnetic body detachably retaining the nucleic acid;
a third plug of oil;
a fourth plug of an eluent that undergoes phase separation from the oil, and elutes the nucleic acid from the magnetic body detachably retaining the nucleic acid; and
a fifth plug of oil,
the first plug, the second plug, the third plug, the fourth plug, and the fifth plug being contained inside the capillary in this order,
wherein the magnetic body detachably retaining the target substance is introduced into the capillary from an end closer to the first plug,
wherein the magnetic body moves into the eluent under a magnetic force externally applied to the capillary,
wherein the target substance elutes from the magnetic body in the eluent, and
wherein the eluent with the eluted target substance moves to an end closer to the fifth plug, and discharges from the capillary at the end closer to the fifth plug.

15. The method according to claim 13, further comprising moving the magnetic body within a plane that crosses the longitudinal direction of the capillary.

16. The method according to claim 13, wherein the liquid with the eluted target substance is moved along the longitudinal direction of the capillary by applying pressure inside the capillary.

17. The method according to claim 16, wherein the pressure is applied with a plunger.

18. The method according to claim 13, wherein the liquid with the eluted target substance is moved along the longitudinal direction of the capillary by creating suction inside the capillary.

19. The method according to claim 18, wherein the suction is created with a vacuum pump.

20. The method according to claim 13,
wherein the capillary has detachable sealing members at the both ends, and
wherein the capillary is vertically held, and the liquid with the eluted target substance is moved along the longitudinal direction of the capillary by opening and closing the sealing members.
